(19) United States
(12) Patent Application Publication
(10) Pub. No.: US 2003/0073234 A1
(43) Pub. Date: Apr. 17, 2003

(54) Clonal human embryonic stem cell lines and methods of generating same

(76) Inventors: Michal Amit, Misgav (IL); James A. Thomson, Madison, WI (US)

Correspondence Address: G. E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 09/975,011

(22) Filed: Oct. 12, 2001

(51) Int. Cl. C12N 5/08; C12Q 1/40

(52) U.S. Cl. 435/366

(57) ABSTRACT

A method of establishing a clonal embryonic stem cell line capable of sustaining a phenotype of normal embryonic stem cells following at least eight months of in vitro culture is disclosed. The method is effected by culturing an individual embryonic stem cell for at least eight months in a serum-free medium, thereby establishing the clonal embryonic stem cell line capable of sustaining said phenotype of normal embryonic stem cells following at least eight months of in vitro culture.

### FIELD AND BACKGROUND OF THE INVENTION

The present invention relates to pluripotent cell lines and methods of generating same. More particularly, the present invention relates to clonal human embryonic stem cell (ESC) lines capable of sustaining a phenotype of normal ESC following at least eight months of in vitro culture and methods of generating same.

Embryonic stem cells, being totipotent, have the potential to develop into any type of cell and to generate any type of tissue, organ or body part including a whole organism. As such, it is expected that the ability to provide normal clonal human ESC on demand and to manipulate the differentiation thereof will provide powerful tool capable of driving radical advances in the biomedical, industrial and scientific fields. Potential applications of ESC are far ranging and include drug discovery and testing, generation of cells, tissues and organs for use in transplantation, production of biomolecules, testing the toxicity and/or teratogenicity of compounds and facilitating the study of developmental and other biological processes. For example, diseases presently expected to be treatable by therapeutic transplantation of ESC or ESC-derived cells include Parkinson's disease, cardiac infarcts, juvenile-onset diabetes mellitus, and leukemia (Gearhart J. Science 1998, 282:1061; Rossant and Nagy, Nature Biotech. 1999, 17:23).

There are, however, significant hurdles to the practical exploitation of human ESC due to ethical and legal constraints severely limiting the obtainment and utilization of human embryos from which human ESC are derived. As a result of these hurdles, current knowledge of the post-implantation human embryo is largely based on a limited number of static histological sections such that the underlying mechanisms controlling the developmental decisions of the early human embryo remain essentially unexplored.

Several prior art approaches have been employed in order to attempt to provide normal ESC, for example, for human biomedical application or for modeling of human or mammalian biology.

One approach has employed non-human tuor cells, such as murine embryonic carcinoma cells. These are pluripotent, immortal cell lines derived from mouse teratocarcinomas which, in turn, are derived from germ cells. However, although germ cells, like ESC, are theoretically totipotent (i.e., capable of forming all cell types in the body), embryonic carcinoma cells possess a limited developmental potential relative to ESC and display the abnormal karyotypes typical of tumor derived lines (Rossant and Papaioannou, Cell Differ 15:155-161, 1984). Furthermore, the use of non-human cells severely limits their application towards human therapy or biological modeling, as described in greater detail hereinbelow.

Another, more advanced, approach has employed human embryonic carcinoma cells (Andrews, et al., Lab. Invest. 50(2):147-162, 1984; Andrews, et al., in: Robertson E., ed. Teratocareinomas and Embryonic Stem Cells: A Practical Approach. Oxford: IRL press, pp. 207-246, 1987). Human embryonic carcinoma cells can be induced to differentiate in culture and the differentiation thereof is characterized by the loss of specific cell surface markers (SSEA-3, SSEA-4, TRA-1-60, and TRA-1-81) and the appearance of new markers (Andrews, et al., 1987, supra). Such embryonic carcinoma cells can form teratocarcinomas containing derivatives of multiple embryonic lineages in tumors in nude mice, however, as with murine embryonic carcinoma cell lines, the range of differentiation of these human cells is limited as compared to that obtained with ESC. Furthermore, as with murine lines, all human embryonic carcinoma cell lines derived to date present the major disadvantage of being aneuploid.

Yet another approach has been to employ pluripotent cell lines derived from embryos of rodents, such as mice, hamsters, rat and rabbits (Bradley, et al., Nature 309:255-256, 1984; Doetschmanet al. Dev Biol 127:224-227, 1988; Evans & Kaufman, Nature 292:154-156, 1981; Evans, et al., Theriogenology 33(1):125-128, 1990; Giles et al. Mol Reprod Dev 36:130138, 1993; Graves & Moreadith, Mol Reprod Dev 36:424-433, 1993; Iannaccone et al. Dev Biol 163:288-292, 1994; Martin, Proc Natl Acad Sci USA 78:7634-7638, 1981; Notarianni, et al., J. Reprod. Fertil. 41(Suppl.):51-56, 1990; Giles, et at., Mol. Reprod. Dev. 36:130-138, 1993; Graves, et al., Mol. Reprod Dev. 36:424-433, 1993; Sukoyan, et al., Mol. Reprod. Dev. 33:418-431, 1992; Sukoyan, et al., Mol. Reprod. Dev. 36:148-158, 1993). The use of such non-human cell lines presents significant and obvious disadvantages, however. For example, with respect to modeling human biological processes; there are significant differences between early mouse and early human development, such as in the extraembryonic membranes, placenta, and in the arrangement of the germ layers at the time of gastrulation. Whereas the yolk sac in murine embryos is a robust, well-vascularized extraembryonic tissue being functionally important throughout gestation, in the human embryo this organ is reduced to a vestigial structure part-way through embryonic development (Kaufman, M. H. (1992). “The Atlas of Mouse Development.” Academic Press, London, O'Rahilly, R., and Muller, F. (1987). “Developmental Stages in Human Embryos,” Carnegie Institution of Washington, Washington, D.C.). Thus, non-human ESC-derived biological materials, being xenogeneic with respect to, being structurally unrelated and/or being functionally unrelated to human biological materials, cannot serve as a satisfactory substitute for human ESC for providing human-compatible or human-homologous biomolecules, cells, tissues and organs, such as for optimal medical application or for realistically modeling human biological processes.

In a further approach, non-human primate polygonal ESC cultures derived from clumps of rhesus macaque or pygmy marmoset embryo cells have also been utilized (Thomson, J. A. et al., (1995) Proc. Natl. Acad. Sci. USA 92, 7844-7849; Thomson, J. A., and Marshall, V. S. (1998) Curr. Top. Dev. Biol. 38, 133-165; U.S. Pat. No. 6,200,806 to Thompson). While such cell lines were shown to be capable, following prolonged in, vitro culture, of normal proliferation in an undifferentiated state, of differentiation to denvatives of all three embryonic germ layers (endoderm, mesoderm, and ectoderm) and of maintaining a normal karotype, such lines are polyclonal, and thus non-phenotypically homogeneous, and, furthermore, being from non-human species, also cannot be satisfactorily employed for providing human-compatible or human-homologous biological materials.

Yet further prior art approaches for overcoming the aforementioned hurdles for obtainment and culture of human ESC have used in vitro cultures of non-clonal human ESC or non-clonal human fetal germ cells. While constituting a marked improvement over the use of non-human cells or of tumor-derived lines for human biomedical application or for modeling human biological processes, such non-clonal cultures, following the long term, may suffer from impaired proliferative capacity, from loss of pluripotentiality, from kaxyotypic instability and/or from inhomogeneity due their non-clonal origin (Reubinoff, B. E. et al., (2000) Nat Biotechnol 18, 399-404; Shamblott, M. J. et al., (1998) Proc. Natl. Acad. Sci. USA 95, 13726-13731; Thomson, J. A. et al., (1998) Science 282, 1145-1147).

There is thus a widely recognized need for, and it would be highly advantageous to have, methods of generating lines of clonal human ESC displaying a normal ESC phenotype following long term culture devoid of the above limitation.

### SUMMARY OF THE INVENTION

According to the present invention there is provided a method of establishing a clonal embryonic stem cell line capable of sustaining a phenotype of normal embryonic stem cells following at least eight months of in vitro culture, the method comprising culturing an individual embryonic stem cell for at least eight months in a serum-free medium, thereby establishing the clonal embryonic stem cell line capable of sustaining the phenotype of normal embryonic stem cells following at least eight months of in vitro culture.

According to further features in preferred embodiments of the invention described below, the individual embryonic stem cell is a human embryonic stem cell.

According to still further features in the described preferred embodiments, the phenotype of normal embryonic stem cells is characterized by a normal karyotype, a non-increasing population doubling time selected from a range of 28 to 42 hours, a non-decreasing telomere length non-decreasing telomerase activity and pluripotentiality.

According to sill further features in the described preferred embodiments, the non-increasing population doubling time is selected from a range of 33 to 37 hours.

According to still further features in the described preferred embodiments, the non-decreasing telomere length is selected from a range of 4 to 16 kb.

According to still further features in the described preferred embodiments, the non-decreasing telomere length is selected from a range of 8 to 12 kb.

According to still further features in the described preferred embodiments, the pluripotentiality is characterized by the capacity to differentiate into endodermal, mesodermal and ectodermal cells.

According to still further features in the described preferred embodiments, the method of establishing a clonal embryonic stem cell line capable of sustaining a phenotype of normal embryonic stem cells following at least eight months of in vitro culture further comprises the step of obtaining the individual embryonic stem cell from a source selected from the group consisting of an embryonic stem cell culture, a blastocyst inner cell mass, a blastocyst, embryonic germ cells, an embryonic germ cell culture, an embryo and a fetus prior to the step of culturing.

According to still further features in the described preferred embodiments, the step of obtaining the individual embryonic stem cell from the blastocyte inner mass is effected by (a) isolating a blastocyst; (b) isolating cells from the inner cell mass of the blastocyst; (c) culturing the cells from the inner cell mass on mouse embryonic feeder fibroblasts, thereby generating an inner cell mass-derived cell mass; (d) dissociating the inner cell mass-derived cell mass into dissociated cells: (e) culturing the dissociated cells on mouse embryonic feeder fibroblasts, thereby generating dissociated cell-derived colonies; (f) selectively harvesting from among the dissociated cell-derived colonies a colony with morphologically compact cells, cells with high nucleus-to-cytoplasm ratio and/or cells with prominent nucleoli; and (g) dissociating the colony with morphologically compact cells, cells with high nucleus-to-cytoplasm ratio and/or cells with prominent nucleoli into individual cells thereby obtaining the individual embryonic stem cell.

According to still further features in the described preferred embodiments, the serum-free medium includes feeder fibroblasts.

According to still fixer features in the described preferred embodiments, the feeder fibroblasts are murine.

According to still further features in the described preferred embodiments, the feeder fibroblasts are embryonic.

According to still further features in the described preferred embodiments, the serum-free medium includes 0.4 to 40 ng/ml bFGF.

According to still further features in the described preferred embodiments, the serum-free medium includes 1 to 16 ng/ml bFGF.

According to still further features in the described preferred embodiments, the serum-free medium includes 2 to 8 ng/ml bFGF.

According to still further features in the described preferred embodiments, the serum-free medium includes 4 ng/ml bFGF.

According to another aspect of the present invention there is provided a clone human embryonic stem cell line being capable of sustaining a normal embryonic stem cell phenotype following at least eight months of in vitro culturing.

According to further features in preferred embodiments of the invention described below, the in vitro culturing is effected on mouse embryonic feeder fibroblasts in serum-free medium supplemented with basic fibroblast growth factor.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purpose of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are photomicrographs depicting H9 human ESC cultured for 14 days in serum-free medium in the presence (4 ng/ml) or absence of bFGF FIGS. 1a and 1b, respectively). ESC plated in serum-free medium in the presence of bFGF continued active, undifferentiated proliferation throughout the culture period. ESC plated in the absence of bFGF uniformly differentiated into a flattened, epithelial morphology by the end of the 14 day culture period. Bar=200 μM.

FIGS. 2a-c are photomicrographs depicting representative metaphase preparations from H9.2 cells at 8 months (PD 175) of continuous culture. Shown are a captured metaphase spectral image (FIG. 2a), classified processed image (FIG. 2b) and reverse DAPI image (FIG. 2c). Karyotype: 46, XX normal female.

FIGS. 3a-c depict telomere length and telomerase activity in human ESC. FIG. 3a is photograph depicting Southern blotting analysis of terminal restriction fragment (TRF) from the H9 parent line and 2 clonal lines, H9.1 and H9.2 at different times in vitro. Passage number for the clones is represented as number of passages following subcloning at passage 29 (p29+X). Cells were passaged at 7 day intervals. MEP indicates irradiated mouse primary embryonic fibroblasts. FIG. 3b is plot depicting mean TRF lengths. Telomere length was quantified using a PhosphorImager and ImageQuant software. The mean TRF length for each lane is an integral function based on the densitometric readings in reference to the standards for each gel (FIG. 3a). Mean TRF length was calculated from replicate analyses: light and dark gray bars, respectively, represent data and replicate analysis of selected samples from the gel depicted in FIG. 3a. FIG. 3c is a photograph depicting TRAP analysis of telomerase activity in human ESC. The TRAP assay is a primer extension assay in which telomerase synthesizes telomeric repeats onto oligonucleotide primers. The telomerase extension products serve as a template for PCR amplification. The laddering in the TRAP gel represents increasing numbers of telomeric repeats in which radionucleotides are incorporated. Samples were run in triplicate with the third sample as a negative control (?) in which cell lysates were heat inactivated prior to the assay. The positive control was a cell extract from the telomerase expressing tumor line H1299.

FIGS. 4a-d are photomicrographs depicting teratomas from H9.1 cells. Approximately 5×10⁶ H9.1 cells that had been cultured for six months (PD 131) following clonal derivation were injected into the hind leg musculature of a SCID-beige mouse. The resulting tumor was harvested and examined three months after injection. FIG. 4a depicts a low power view of a field exhibiting differentiated derivatives of all three embryonic germ layers. Areas enclosed in boxes are enlarged in FIGS. 4b-c (bar=500 μM). FIG. 4b depicts gut epithelium with adjacent smooth muscle (bar=100 μM). FIG. 4c depicts embryonic glomeruli and renal tubules (bar=100 μM). FIG. 4d depicts neural epithelium (bar=100 μM).

### DESCRIPTION OF THE PREFERRED EMBODIMENTS

The present invention is of clonal embryonic stem cell lines capable of sustaining a phenotype of normal embryonic stem cells following at least eight months of in vitro culture and methods of establishing same. Specifically, the present invention relates to methods of conveniently producing unlimited numbers of normal clonal, preferably human ESC, which unlike the non-clonal prior art human ESC lines, display phenotypic homogeneity, pluripotentiality, a normal karyotype, a normal and stable population doubling (PD) time and a potentially unlimited proliferative capacity following long term in vitro culture. As such, the ESC lines of the present invention have the unlimited capacity to provide normal clonal ESC having the capacity to grow and differentiate into any desired biological material, such as biomolecules, cells, tissues, organs, body parts or whole organisms, which biological materials being optimal for use in biomedical, scientific and industrial applications.

The principles and operation of the present invention may be better understood with reference to the accompanying descriptions and Examples.

Before explaining at least one embodiment of the invention in detail, it is to be understood that the invention is not limited in its application to the details of construction and the arrangement of the components set forth in the following description or exemplified in the Examples. The invention is capable of other embodiments or of being practiced or carried out in various ways. Also, it is to be understood that the phraseology and terminology employed herein is for the purpose of description and should not be regarded as limiting.

Various methods of providing ESC, such as human ESC, have been described by the prior art.

For example, human ESC can be directly isolated from human embryos, however, this method cannot be generally employed due to legal and ethical obstacles regarding obtainment and utilization of human embryos and, in any case, only limited numbers of cells can be directly obtained via such methods. Another approach has employed establishment of non-clonal lines of human ESC derived from a plurality of human blastocyst inner cell mass cells to provide non-clonal lines of human ESC. Such prior art cell lines, being non-clonal, however, do not provide well defined, phenotypically homogeneous populations of cells and are therefore not optimal for use in human biomedical, scientific or industrial applications.

Thus, all prior art approaches have failed to provide adequate solutions for providing human ESC lines displaying phenotypic homogeneity, pluripotentiality, a normal karyotype, a normal and stable population doubling time and a potentially unlimited proliferative capacity following long term in vitro culture, which ESC lines being optimal for biomedical, industrial or scientific use.

While reducing the present invention to practice it was uncovered that culturing individual human ESC under suitable serum-free conditions could generate clonal human ESC lines possessing a stable, homogenous and normal ESC phenotype following long term in vitro culture.

Thus, according to one aspect of the present invention, there is provided a method of establishing clonal ESC lines being capable of sustaining a phenotype of normal ESC following at least eight months of in vitro culture.

The clonal ESC line is etablished by culturing an individual embryonic stem cell which is obtained from an embryonic stem cell culture, a blastocyst inner cell mass, a blastocyst, embryonic germ cells, an embryonic germ cell culture, an embryo or a fetus. Example 1 of the Examples section which follows illustrates isolation of an individual human embryonic stem cell from a blastocyst inner cell mass.

Culturing of the individual embryonic stem cell is effected in serum-free medium.

Preferably, the individual embryonic stem cell is cultured on mouse embryonic feeder fibroblasts in a suitable serum-free medium supplemented with bFGF.

Supplementation with bFGF is effected with 0.4 to 40 ng/ml bFGF, preferably with 1 to 16 ng/ml bFGF, more preferably with 2 to 8 ng/ml bFGF or, most preferably, with 4 ng/ml bFGF.

Further description of culturing media and conditions is provided in the Examples section which follows.

Since serum poorly supports ESC cloning efficiency and since serum batches vary widely, replacing serum with defined components, as described in the Examples section which follows, the present method increases efficiency and reduces variability of ESC cloning relative to prior art serum-utilizing methods of establishing ESC lines.

The clonal character of the human ESC line of the present invention represents an improvement over the non-clonal prior art human ESC lines since the former display a more clearly defined, stable and homogeneous phenotype relative to prior art human ESC lines. As such, the ESC line of the present invention, is more suitable for human biomedical, industrial or scientific application than prior art ESC lines.

The ESCs of the present invention display a phenotype of normal ESCs. Such a phenotype is characterized by, for example, a normal karyotype, non-increasing population doubling time, a non-decreasing telomere length, non-decreasing telomerase activity and pluripotentiality following at least eight months of in vitro culture after clonal derivation, as clearly demonstrated in Example 1 of the Examples section, below.

A phenotype characteristic of normal human ESCs can be further characterized by expression of alkaline phosphatase, and the glycolipid cell surface markers known as stage-specific embryonic antigens SSEA-3 and SSEA-4, and the markers TRA-1-60 and TRA-1-81. Antibodies for the SSEA markers are available from the Developmental Studies Hybridoma Bank of the National Institute of Child Health and Human Development. The cell surface markers referred to as TRA-1-60 and TRA-1-81 designate antibodies from hybridomas developed by Peter Andrews of the University of Sheffield and are described in Andrews et al., “Cell lines from human germ cell tumors,” In: Robertson E., ed. Teratocarcinomas and Embryonic Stem Cells. A Practical Approach. Oxford: IRL Press, 207-246, 1987. Alternatively, it should also be understood that other antibodies for these same cell surface markers can be generated.

Normal karyotype determination can be performed by one of ordinary skill in the art, for example, with a standard G-banding technique.

As is further illustrated in the Examples section which follows, the ESCs of the present invention display non-increasing population doubling times which range between 28 to 42 hours or, more preferably, between 33 to 37 hours.

The ESCs of the present invention also display non-decreasing telomere lengths which range between 4 to 16 kb or, more preferably, between 8 to 12 kb, as described in the Example 1 of the Examples section which follows.

The non-decreasing telomere length and non-decreasing telomerase activity of the ESCs of the present invention indicates that these cells possess a potentially unlimited replication capacity since progressive telomere shortening is known, to those well versed in the art, to signal cellular senescence (Harley, C. B. et al., (1990) Nature 345, 458-60; Vaziri, H. et al., (1993) Am. J. Hum. Genet. 52, 661-667). Also, the stable telomerase activity of the ESCs of the present invention is widely recognized, by those well versed in the art, to prevent cellular senescence via maintenance of telomere length (Bodnar, A. G. et al., (1998) Science 279, 34; Jiang, X. R. et al., (1999) Nature Genet. 21, 1114). Furthermore, unlike prior art ESC lines the FSC line of the present invention can be classified as immortal since continued culture proliferation for longer than one year constitutes evidence of immortality (Freshney, Culture of animal cells. New York: Wiley-Liss, 1994) and since as described in the Examples section, the ESC line of the present invention can be cultured, without evidence of senescence, for at least 14 months; 6 months as a non-clonal parental culture followed by 8 months of post-clonal derivation culture.

Thus, tee ESC lines of the present invention constitute an unlimited source of ESCs for use in all applications, including, for example, biomedical, industrial and scientific applications.

Since the ESCs of the ESC lines of the present invention retain their pluripotency, such cells can be induced to differentiate into endodermal, mesodermal and ectodermal cells, as is further described in Example 1 of the Examples section which follows.

By simply altering culturing conditions, the ESCs of the present invention can be induced to differentiate into endodennal cells, tissues or organs such as but not limited to, gut or respiratory epithelial cells or hepatic cells; mesodermal cells, tissues or organs, such as, but not limited to, kidney cells, striated muscle, smooth muscle, bone and cartilage; or ectodermal cells, tissues or organs, such as, but not limited to, nerve cells & glial cells, hair follicles and teeth.

Thus, the capacity of the ESC lines of the present invention to differentiate into any given cell type can be employed to generate cells producing any desired biomolecule.

As illustrated in Example 1 of the Examples section which follows, the clonal human ESCs of the present invention can also be induced to differentiate into cells of the endodemal, mesodermal and ectodermal lineages in an in vivo context, as a teratoma containing cells of all three lineages.

The pluripotentiality of the ESCs of the present invention can be assessed by injecting approirtely 0.5-1.0×10⁶ ESCs into rear leg muscle of 8-12 week old male SCID mice, The resultig teratomas can be fixed in 4% paraformaldehyde and examined histologically for presence of the aforementioned cell and tissue types following paraffin embedding at 8-16 weeks of development.

The differentiated products of the ESC line of the present invention including cells, tissues, organs or body pars, can be used in, for example, therapeutic or prophylactic transplantation, such as, for example, to replace or supplement cells, tissues, organs or body pants impaired or lost due to disease, injury, ageing or accidents. Alternately, applied uses of such cells, tissues, organs or body parts include transplantation in the context of cosmetic or other non-therapeutic applications, such as, for example, hair replacement or other types of cosmetic procedures.

For example, therapeutic transplantation of ESC, such as those of the present invention, has been shown to be highly applicable for treatment of diseases such as neurological, cardiovascular, pancreatic, and hematopoietic diseases (Gearhart J. Science 1998, 282:1061; Rossant and Nagy, Nature Biotech. 1999, 17:23).

The biomolecules, cells, tissues, organs or body parts, derived from the ESC line generated by the method of the present invention can be applied towards scientific uses including, for example, identification of gene targets for pharmacological or therapeutic compounds, testing the toxicity or teratogenicy of compounds, for generating transgenic or chimeric organisms to serve as, for example, models of specific human genetic diseases, for studying differentiation, development or other biological processes.

Chimeras between preimplantation embryos and ESC can be formed, for example, by microinjection of 10-15 ESC into the cavity of a blastocyst. Alternatively, aggregation chimeras can be formed by co-culturing morulae on a lawn of ESC and allowing these to aggregate. Tetraploid chimeras can be formed by aggregating 10-15 ESCs with tetraploid morulae obtained by electrofusion of 2-cell embryos, or incubation of morulae in the cytoskeletal inhibitor cholchicine. Such chimeras can be returned to the uterus of a suitable host and allowed to develop. These ESC will contribute to normal differentiated tissues derived from all three embryonic germ layers and to germ cells. Because ESC can be genetically manipulated prior to chimera formation by standard techniques, transgenic chimera formation followed by embryo transfer can lead to the production of chimeric transgenic organisms.

It will be evident to one well skilled in the art that the method of the present invention can be readily applied to establish non-human normal clonal ESC lines having a phenotype of normal ESCs following long term culture. For example, it has been demonstrated that it is possible to successfully establish rhesus monkey and common marmoset ESC lines using similar conditions (U.S. Pat. No. 6,200,806 to Thomson). Since the evolutionary distance between these two species is far greater than that separating humans and rhesus monkeys and since feeder-dependent human embryonic carcinoma cell lines and primate ESC lines can be established in under highly similar conditions, the method of the present invention can therefore be easily employed to etablish normal clonal non-human primate ESC lines, such as, for example, rhesus monkey or common marmoset ESC lines, having a phenotype of normal ESCs following long term culture.

Additional objects, advantages, and novel features of the present invention will become apparent to one ordinarily skilled in the art upon examination of the following examples, which are not intended to be limiting Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “(Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co,, New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Establishment of Clonal Human ESC Lines Sustaining a Normal ESC Phenotype Following Long Term Culture

The provision and manipulation of human ESC promises to yield major biomedical, industrial and scientific advances. Culturing ESC in vitro has been attempted in the prior art as a means to provide a convenient source of such cells. These approaches, however, have failed to provide clonal human ESC lines displaying a normal ESC phenotype following long term culture. This represents a serious drawback since the optimal use of human ESC for scientific and therapeutic applications critically depends on the ability to generate clonal human ESC lines displaying normal ESC proliferative capacity, a euploid karyotype and an unrestricted developmental potential following long-term culture. Therefore, while reducing the present invention to practice, ESO lines fulfilling such criteria were generated, as described below and as described in Amit, M. et al., Developmental Biology 227, 271-278 (2000).

Materials and Methods:

Generation of clonal human ESC lines: The derivation, routine culture, and characterization of the human ESC line H9 was performed as previously described (Thomson, J. A. et al., (1998) Science 282, 1145-1147). After six months (PD 122) of culture, H9 cells were dissociated to single cells for 7 min with 0.05% trypsin/0.25% EDTA, washed by centrifugation, and plated on mouse embryonic fibroblasts (10⁵ ESC in triplicate wells of 6-well plates) mitotically inactivated by gamma irradiation (35 Gy). To generate single cell derived clonal ESC lines, individual cells were selected by direct observation under a stereomicroscope and transferred by micropipette to individual wells of a 96 well plate containing mouse embryonic fibroblast feeder cells with medium containing 20% serum replacer and 4 ng/ml bFGF. Clones were expanded by routine passage every 7 days with 1 mg/ml collagenase type IV (Gibco BRL, Rockville, Md.) and maintained continuously in medium supplemented with serum replacer and bFGF. Cell were cultured in medium containing 80% “KnockOut” Dulbecco's modified Eagle's medium (DMEM), an optimized medium for mouse ESC (Gibeo BRL, Rockvile, Md.), 1 mM L-Glutamine, 0.1 mM ?-mercaptoethanol, and 1% nonessential amino acids stock (Gibco BRL, Rockville, Md.). This medium was supplemented with either 20% fetal bovine serum (HyClone, Logan, Utah) or 20% KnockOut SR, a serum replacer optimized for mouse ESC (Gibco BRL, Rockville, Md.). The components of KnockOut SR have been previously described (Price, P. J. et al., (1998) International Patent Application WO98/30679). In initial cloning experiments, medium was supplemented with either serum or serum replacer, and either with or without human recombinant bFGF (4 ng/ml). For prolonged culture, the serum-free medium required supplementation with bFGF.

Because ethical considerations in the U.S. do not allow the recovery of human in vivo fertilized preimplantation blastocysts from the uterus, human blastocysts that are derived from preimplantation embryos are derived from in vitro fertilized (IVF) embryos. Experiments on unused (spare) human IVF-produced embryos are allowed in many countries, such as Singapore and the United Kingdom, if the embryos are less than 14 days old. Only high quality embryos are suitable for ES isolation.

Karyotype analysis: Either standard G banding or Multicolor Spectral Karyotyping (SKY) was performed (Schrock, E. et al., (1996) Science 273, 494-7). For SKY analysis, the SKY H-10 kit was used according to the manufacturer's instructions (Applied Spectral Imaging, Inc, Carlsbad, Calif.). Metaphase figures were dropped onto clean glass slides and treated with combinatorially labelled whole genome painting probes. After stringent washes in 50% formamide, images of metaphase spreads were captured using the Applied Spectral Imaging spectrophotometer and SKY software on a Zeiss Axioplan II microscope. Karyotypes were analyzed and arranged with combined software processing of the image and reverse DAPI banding. For each SKY sample, five metaphases images were captured and fully analyzed and twenty metaphase images were captured for modal number determination.

Determination of proliferation rate: Ten separate determinations of population doubling time were performed on the H9 parental line and in the H9.1 and H9.2 clonal lines.

Measurement of telomerase activity and telomere length: ESC expressing TRA-1-60 (a marker of undifferentiated human ESC) were selected from cultures grown on irradiated mouse embryonic fibroblasts. Cells were dissociated using 0.2% EDTA and then incubated with a monoclonal antibody against TRA-1-60 (gift of Peter Andrews). After washing, cells were incubated with goat anti-mouse IgM-conjugated magnetic microbeads and processed through a MACS magnetic separation column (Miltenyi Biotec, Bergisch Gladbach, Germany; Walz, T. M. et al., (1995) Blood 85, 2385-2392). In the samples used for these experiments, greater than 90% of the population was positive for TRA-1-60 using flow cytometric analysis. Telomerase activity was measured using the telomeric repeat amplification protocol (TRAP) assay as described (Kim, N. W. et al., (1994) Science 266, 2011-5; Weinrich, S. L. et al., (1997) Nature Genet. 17, 498-502). Terminal restriction fragment (TRF) size was determined using Southern hybridization essentially as described (Alsopp, R. C. et al., (1992) Proc. Natl. Acad. Sci. USA 89, 10114-10118,; Harley, C. B. et al., (1990) Nature 345, 458-60; Vaziri, H. et al., (1993) Am. J. Hum. Genet. 52, 661-667).

Teratoma formation: H9.1 and H9.2 cells, cultured for six months after cloning and displaying normal karyotypes, were injected into the rear leg muscle of 4-week-old male SCID-beige mice (eight mice total). Cell numbers ranged from 2.5×10⁶ cells to 7.5×10⁶ cells per injection. Three to four months after injection the mice were sacrificed and the resulting teratomas examined histologically.

Results:

Clonal human ESC lines derived from individual human ESC displaying long term pluripotency, normal karyotype and replicative immortality following over 1 year of in vitro culture were generated.

Cloning efficiency: The cloning efficiency of human ESC was extremely poor in previously described culture conditions that included serum. A several-fold increase in cloning efficiency of human ESC was consistently observed when serum-free medium was used instead of serum-containing medium (Table 1).

TABLE 1
Cloning Efficiency of H9 Human ES Cells^{a}

| | (−) bFGF | (+) bFGF (4 ng/ml) |
|---|---|---|
| 20% Serum | 240 ± 28 (0.24)^{a} | 260 ± 12 (0.26)^{a} |
| 20% Serum Replacer | 633 ± 43 (0.63)^{†} | 826 ± 61 (0.83)^{‡} |

^{a}Values are expressed as the mean number of colonies per 10⁵ ESC plated ± SE (figures in parentheses represent percent colony cloning efficiency).
^{a,†,‡}Different superscripts differ significantly (p < 0.05), Tukey-Kramer HSD.

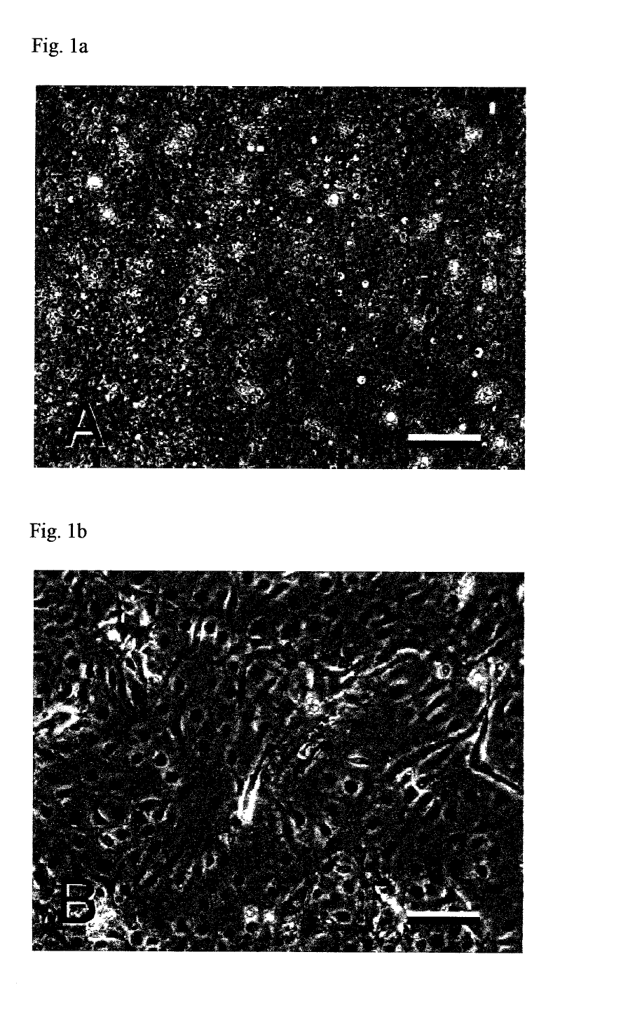

The addition of bFGF to the medium altered the morphology of human ESC, resulted in smaller cells in tighter colonies (FIG. 1a). The long term culture of human ESC in the presence of serum did not require the addition of exogenous bPGF and the addition of bFGF to serum-containing medium did not significantly increase human ESC cloning efficiency (Table 1). However, in serum-free medium, addition of bFGF increased the initial cloning efficiency of human ESC and bFGF was found to be required for continued, undifferentiated proliferation. In serum-free medium lacking bFGF, human ESC became uniformly differentiated by two weeks post-plating (FIG. 1b).

Generation of clonal human ESC lines. Of 384 H9 cells individually plated per well in 96-well plates, two clones were successflly expanded (H9.1 and H9.2). Both of these clones were subsequently cultured continuously in medium supplemented with serum replacer and bFGF.

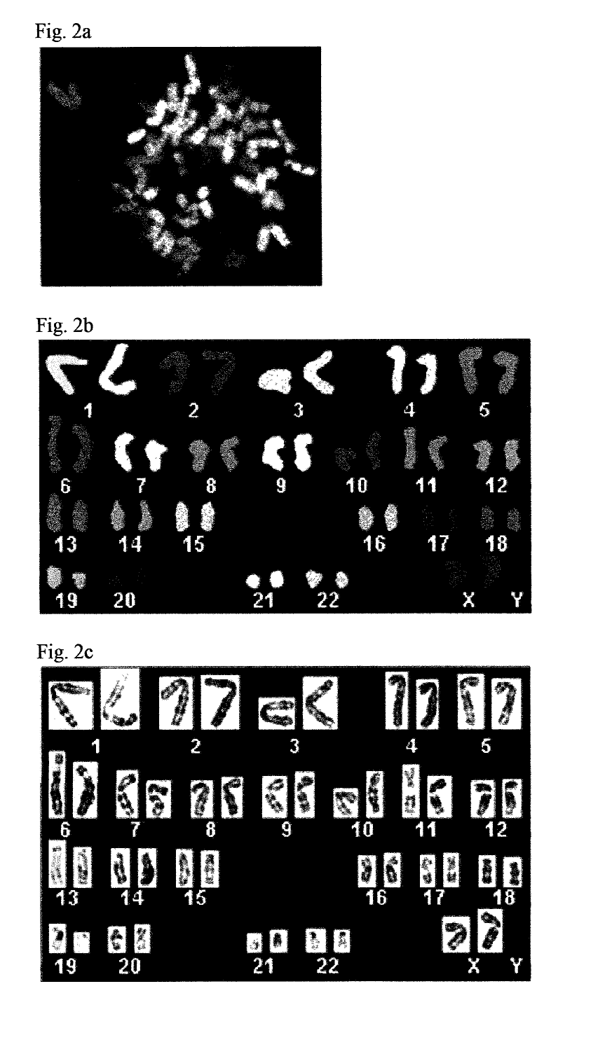

Long term maintenance of normal karyotype by clonal human ESC lines: Clonal lines H9.1 and H9.2 were found to display a normal XX karyotype after 8 months, or more of continuous culture following clonal derivation from H9 cells at PD122 (FIGS. 2a-c). Although, evaluation of karyotype in the H9 parental population six months after its derivation (PD 122) exhibited a normal XX karyotppe by standard G-banding techniques (20 chomosomal spreads analyzed), seven months after its derivation, in a single karyotype preparation, 4/20 spreads demonstrated random abnormalities; one with a translocation to chromosome 13 short arm, one with an inverted chromosome 20, one with a translocation to the number 4 short arm, and one with multiple fragmentation (data not shown). Subsequently, at 8, 10, and 12.75 months after derivation (PD 260), H9 cells exhibited normal karyotypes in all 20 chromosomal spreads examined.

Long term retention of normal ESC proliferation capacity by clonal human ESC lines: Population doubling time was measured in the H9 parental line and in both H9.1 and H9.2 clonal lines and no significant differences were found between the parental H9 line and the clonal H9.1 and H9.2 lines. The average population doubling time for ten separate determinations was 35.3±2.0 hours (mean±standard error of the mean). Because of the considerable cell death observed in these human ESC cultures, this population doubling time may underestimate the replication rate of the ESC that survive.

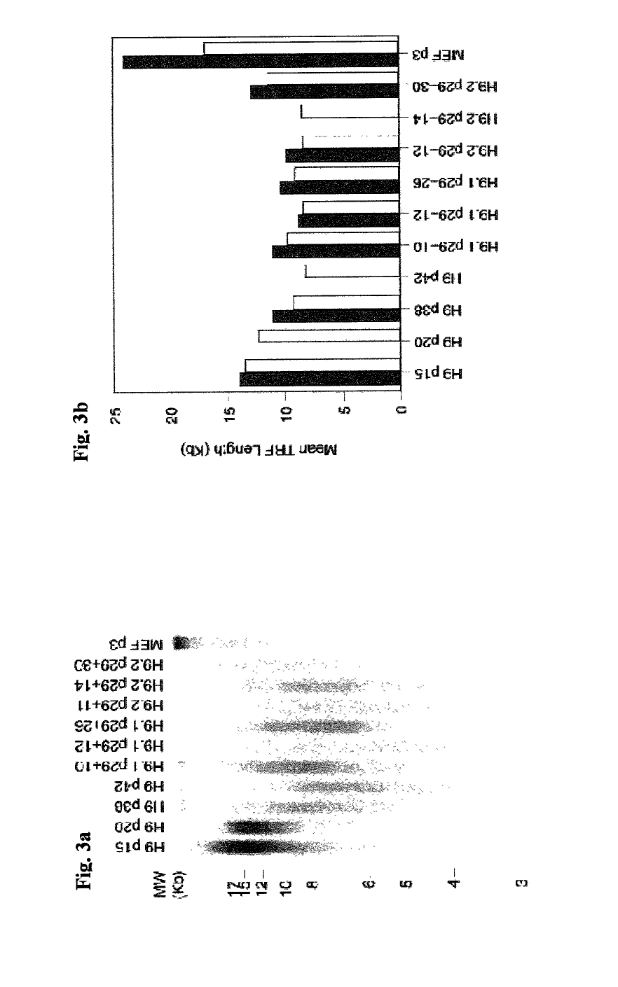

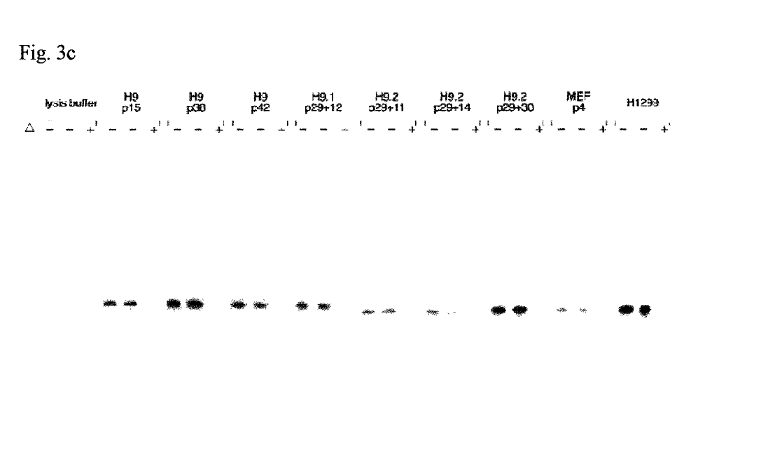

Long term retention of high telomerase activity in clonal human ESC lines: Telomerase activity was found to remain high in H9, H9.1, and H9.2 cells at all time points examined (FIGS. 3a-c), ranging from 52-196% of that found in H1299, a lung tunor cell line. In these experiments, the human ESC were separated from fibroblast feeders by magnetic bead sorting to greater than 90% of the cells as determined by Tra-1-60 expression. The telomerase activity of the MEF (FIGS. 3a-c) was negligible, being only 4-15% of that found in human ESC.

In general, the presence of telomerase activity is associated with a maintenance of telomere length. H9.1 and H9.2 clones showed telomere lengths stabilized between 8-12 kb. As seen in other populations of telomerase-positive cells, changes in telomere length did not correlate with telomerase activity. At PD 57 after clonal derivation, H9.2 cells had a telomere length of about 8 kb which increased to 13 kb by PD 143. H9.1 cells showed an initial decrease in telomere length followed by an increase to about 11 kb at PD 123. On the other hand, the parental cell line, H9, demonstrated an initial decrease in telomere length from 14 kb at PD 71 (passage 15) to 9 kb at PD 200 (passage 42) (FIGS. 3a-c). Cells at later PD will need to be examined to determine if telomeres continue to shorten. However, H9 was still proliferating well at PD 304.

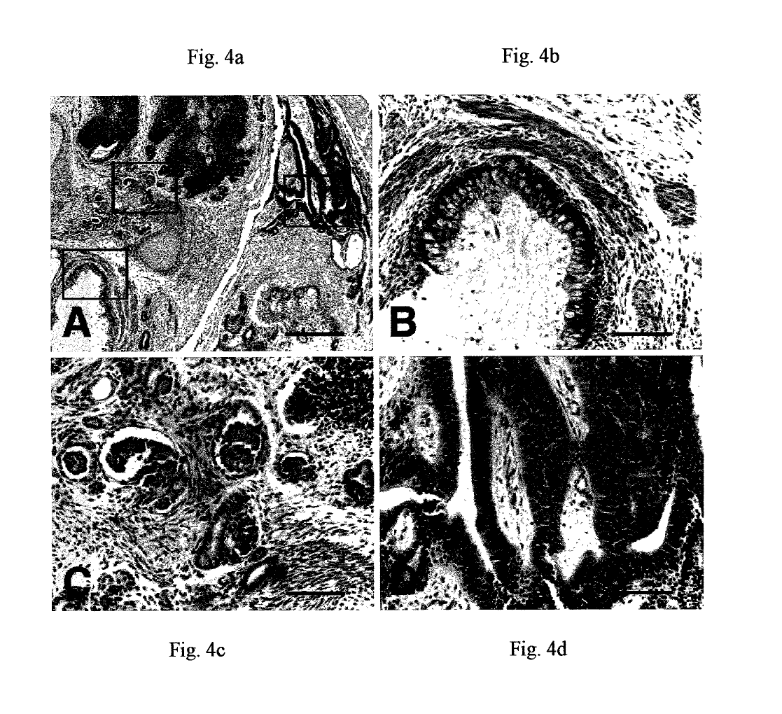

Long term retention of pluripotentiality: The H9.1 and H9.2 clones maintained the potential to form derivatives of all three embryonic germ layers after long term culture in serum-free conditions. After 6 months of culture (PD 122), H9.1 and H9.2 clones injected in vivo formed teratomas containing derivatives of all three embryonic germ layers, including gut epithelium (endoderm); embryonic kidney, striated muscle, smooth muscle, bone, cartilage (mesoderm); and neural tissue (ectoderm) (FIGS. 4a-d). The range of differentiation observed with the teratomas of the high passage H9.1 and H9.2 cells was comparable to that observed in teratomas formed by low passage parental 19 cells.

Conclusion:

The present invention demonstrates the generation of clonal human ESC lines displaying germ layer pluripotentiality, a normal karyotype and a normal ESC replication capacity in terms of replication rate, telomere length and telomerase activity following long tern culture. As such the method of the present invention represents a marked improvement over prior art methods of providing human ESC. The ESC of the present invention therefore constitute an optimal and convenient means of readily providing unlimited numbers of normal clonal human ESC for biomedical, industrial and scientific application.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the sane extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of establishing a clonal embryonic stem cell line capable of sustaining a phenotype of normal embryonic stem cells following at least eight months of in vitro culturing, the method comprising culturing an individual embryonic stem cell for at least eight months in a serum-free medium, thereby establishing the clonal embryonic stem cell line capable of sustaining said phenotype of normal embryonic stem cells following at least eight months of in vitro culture.

2. The method of claim 1, wherein said individual embryonic stem cell is a human embryonic stem cell.

3. The method of claim 1, wherein the phenotype of normal embryonic stem cells is characterized by a normal karyotype, a non-increasing population doubling time selected from a range of 28 to 42 hours, a non-decreasing telomere length, non-decreasing telomerase activity and pluripotentiality.

4. The method of claim 3, wherein said non-increasing population doubling time is selected from a range of 33 to 37 hours.

5. The method of claim 3, wherein said non-decreasing telomere length is selected from a range of 4 to 16 kb.

6. The method of claim 37 wherein said non-decreasing telomere length is selected from a range of 8 to 12 kb.

7. The method of claim 3, wherein said pluripotentiality is characterized by the capacity to differentiate into endodermal, mesodermal and ectodermal cells.

8. The method of claim 1, further comprising the step of obtaining said individual embryonic stem cell from a source selected from the group consisting of an embryonic stem cell culture, a blastocyst inner cell mass, a blastocyst, embryonic germ cells, an embryonic germ cell culture, an embryo and a fetus prior to said step of culturing.

9. The method of claim 8, wherein said step of obtaining said individual embryonic stem cell from said blastocyte inner mass is effected by:
(a) isolating a blastocyst;
(b) isolating cells from the inner cell mass of said blastocyst;
(c) culturing said cells from the inner cell mass on mouse embryonic feeder fibroblasts, hereby generating an inner cell mass-derived cell mass;
(d) dissociating said inner cell mass-derived cell mass into dissociated cells;
(e) culturing said dissociated cells on mouse embryonic feeder fibroblasts, thereby generating dissociated cell-derived colonies;
(f) selectively harvesting from among said dissociated cell-derived colonies a colony with morphologically compact cells, cells with high nucleus-to-cytoplasm ratio and/or cells with prominent nucleoli; and
(g) dissociating said colony with morphologically compact cells, cells with high nucleus-to-cytoplasm ratio and/or cells with prominent nucleoli into individual cells thereby obtaining said individual embryonic stem cell.

10. The method of claim 1, wherein said serum-free medium includes feeder fibroblasts.

11. The method of claim 10, wherein said feeder fibroblasts are murine.

12. The method of claim 10, wherein said feeder fibroblasts are embryonic.

13. The method of claim 1, wherein said serum-free medium includes 0.4 to 40 ng/ml bFGF.

14. The method of claim 1, wherein said serum-free medium includes 1 to 16 ng/ml bFGF.

15. The method of claim 1, wherein said serum-free medium includes 2 to 8 ng/ml bFGF.

16. The method of claim 1, wherein said serum-free medium includes 4 ng/ml bFGF.

17. A clonal human embryonic stem cell line being capable of sustaining a normal embryonic stem cell phenotype following at least eight months of in vitro culturing.

18. The clonal human embyonic stem cell line of claim 17, wherein said in vitro culturing is effected on mouse embryonic feeder fibroblasts in serum-free medium supplemented with basic fibroblast growth factor.

19. The clonal human embryonic stem cell line of claim 17, wherein the phenotype of normal embryonic stem cells is characterized by a normal karyotype, a non-increasing population doubling time selected from a range of 28 to 42 hours, a non-decreasing telomere length, non-decreasing telomerase activity and pluripotentiality.

20. The clonal human embryonic stem cell line of claim 17, wherein said non-increasing population doubling time is selected from a range of 33 to 37 hours.

21. The clonal human embyonic stem cell line of claim 17, wherein said non-decreasing telomere length is selected frown a range of 4 to 16 kb.

22. The clonal human embryonic stem cell line of claim 17, wherein said non-decreasing telomere length is selected from a range of 8 to 12 kb.

23. The clonal human embyonic stem cell line of claim 17, wherein said pluripotentiality is characterized by the capacity to differentiate into endodermal, mesodermal and ectodermal cells.

24. A clonal human embryonic stem cell line being capable of sustaining a normal embryonic stem cell phenotype following at least twelve months of in vitro culturing.
